(19) United States
(12) Patent Application Publication
(10) Pub. No.: US 2013/0273635 A1
(43) Pub. Date: Oct. 17, 2013

(54) BACTERIOPHAGE KILLING PSEUDOMONAS AERUGINOSA AND STAPHYLOCOCCUS AUREUS

(71) Applicants: Jung Min Kim, Daegu (KR); Shuk Ho Kim, Daegu (KR); Seongjun Yoon, Seoul (KR); Sooyoun Jun, Seoul (KR); Sanghyeon Kang, Seoul (KR)

(72) Inventors: Jung Min Kim, Daegu (KR); Shuk Ho Kim, Daegu (KR); Seongjun Yoon, Seoul (KR); Sooyoun Jun, Seoul (KR); Sanghyeon Kang, Seoul (KR)

(73) Assignee: INTRON BIOTECHNOLOGY, INC., Kyungki-Do (KR)

(21) Appl. No.: 13/445,564

(22) Filed: Apr. 12, 2012

(51) Int. Cl. C12N 7/00

(52) U.S. Cl. 435/235.1

(57) ABSTRACT

The present invention relates to a bacteriophage PA1Φ belonging to family Siphoviridae, characterized that it is capable of killing one or more bacteria strains selected from a group comprising Pseudomonas aeruginosa, Staphylococcus aureus, Staphylococcus epidermidis, Staphylococcus homonis, Shigella sonnei, Listeria monocytogenes and Streptococcus pneumonia, and contains a full-length genome of SEQ ID NO: 1.

According to the present invention, the bacteriophage PA1Φ can be used to kill said bacteria and reduce the same effectively. Also, it can be used to remove biofilms generated by said bacteria. Especially, this bacteriophage is applicable for medical industry, food industry, biotechnology and the like, because it is a sort of virus that kills host bacteria without any adverse effect on human, animals and so on. In addition, this bacteriophage can kill noxious bacteria on target sites or target supports without any problem related with resistance development of bacteria.

### TECHNICAL FIELD

The present invention relates to a bacteriophage killing Pseudomonas aeruginosa and Staphylococcus aureus, more particularly to the bacteriophage PA1Φ belonging to family Siphoviridae, characterized that it is capable of killing Pseudomonas aeruginosa and Staphylococcus aureus and contains a full-length genome of SEQ ID NO: 1.

### BACKGROUND ART

Pseudomonas aeruginosa is a general Gram-negative rod causing an infectious disease in animals including human and usually exists in a variety of environments including soil, swamp, human skins. It is one of the most common opportunistic pathogens, resulting in inflammations and septicemia in immuno-compromised patients. Presently, Pseudomonas aeruginosa is known as a main bacterium causing acquired infections in a hospital. Especially in foreign countries, it is highly prevalent among patients suffering from cystic fibrosis and further, these patients may be fatally affected by this infection.

Staphylococcus aureus is another bacterium causing acquired infections in a hospital. It is an indigenous bacterium often found even among normal people and around natural environment. This bacterium may attack injured sites, debilitated patients and surgical wounds generated during an operation by contaminated medical devices and the like. This infection results in purulent diseases that are hardly treated due to anti-bacterial resistance. Staphylococcus aureus has a feature to survive for a long time while being attached onto the surface of medical devices or non-living bodies. Therefore, it is difficult to remove them completely with conventional detergents or alcohols. This is referred to as the formation of bacterial biofilm. Biofilms resist antibiotic treatment and biofilm bacteria show much greater resistance to antibiotics than their free-living (planktonic) counterparts. Therefore, it is not expected to eradicate them sufficiently only by conventional antibiotic treatment. Problematically, new antibiotic-resistant bacteria has continued to appear and expand and further, super-bacteria resisting against almost all kinds of anti-bacterial therapeutics are being found. Hence, it is anticipated and urgently required to develop alternative medicines in place of existing anti-bacterial agents.

Bacteriophage is a virus that infects bacteria specifically and lyses them. Since discovered first in 1915, it has been investigated to treat lots of bacterial diseases. Firstly, Felix d'Herelle, a discoverer of a bacteriophage has attempted 80 years ago to apply the bacteriophage for therapeutic use in infectious diseases.

From the past to the present, the therapeutic uses of bacteriophages have been researched in Georgia, Poland and the old Soviet Union consistently. In practice, they are being utilized to treat pathogenic microorganisms. In 1994, antibiotic-resistant E. coli infected to a recurrent diaphragm tumor after a gastrectomy, was been cleared perfectly by using the bacteriophage. In 1995, 46 patients suffering from acute and chronic urinary inflammations were treated with the bacteriophage, so that 92% of patients could be treated clinically and pathogenic microorganisms could be eliminated clearly from 84% of patients. In 1999, Klebsiella pneumoniae causing meningitis in neonates was treated successfully by injecting bacteriophages orally, even though having failed to eradicate this by using antibiotics. Also in 2001, the bacteriophage was administered orally to 20 of patients suffering from intractable tumors three times a day during 2 to 9-week period, so as to treat these patients successfully. In 2001, 54 of burn patients were protected from infections of Staphylococcus, Streptococcus, Proteus and the like by treating bacteriophages in 1.5 to 2-folds more than infected bacteria on burn lesions. In 2002, the mixture of bacteriophage and bio-degradable material was administered on lesions and in tumors, so that 67 patients among 96 of total patients could be restored.

Recently, it is attempted to screen bacteriophages exhibiting the broad anti-bacterial spectrum and further, to accomplish the pharmacodynamic studies upon the delivery of anti-bacterial drugs regarding their efficacies. Accordingly, the applicability of the bacteriophages is being increased. Unfortunately, the studies upon bacteriophages are being performed actively in foreign countries, but they have just started domestically.

The researches upon bacteriophage therapies have been introduced in worldwide academic journals, Nature Review and Nature Bacteriology. In particular, the method of screening bacteriophages for therapeutic use and phage therapy were described in detail.

In 2007, US Food & Drug Administration (FDA) has approved a Stage I Clinical test for bacteriophage use in order to treat the bacterial infection in diabetic necrosis of feet. This test enables the bacteriophages applied for therapeutic purpose to treat infectious diseases of human as well as those of animals. Furthermore, in 2008, FDA has approved the method of reducing Salmonella contamination during processing chickens by using a Salmonella specific bacteriophage. In order to treat infectious diseases, it is necessary to screen bactericidal factors originated from bacteriophages as well as to isolate new bacteriophages. These are expected to contribute to the treatment of infectious diseases economically and to improve health care systems. Therefore, it is required to develop related technologies and reform research environment by national supports on this theme.

### DISCLOSURE

### Technical Problem

Hence, in order to overcome the above-mentioned problems of infectious diseases, the present inventors have tried to isolate the bacteriophage PA1Φ from natural environment, and elucidated that the bacteriophage PA1Φ is capable of killing Pseudomonas aeruginosa and Staphylococcus aureus effectively and removes biofilms generated by the same efficiency. As a consequence, we have completed the present invention successfully.

Accordingly, it is the main object of the present invention to provide a bacteriophage PA1Φ capable of killing Pseudomonas aeruginosa and Staphylococcus aureus.

It is another object of the present invention to provide a pharmaceutical composition comprising the bacteriophage PA1Φ as an effective ingredient, which can be used to treat diseases caused by the bacteria described above.

It is the other object of the present invention to provide an antibiotic and a disinfectant comprising the bacteriophage PA1Φ as an effective ingredient.

### Technical Solution

To achieve the above objects, according to one embodiment, the present invention provides the bacteriophage PA1Φ belonging to family Siphoviridae, characterized that it is capable of killing Pseudomonas aeruginosa and Staphylococcus aureus and contains a full-length genome of SEQ ID NO: 1.

According to the present invention, the bacteriophage PA1Φ was first discovered, and identified by the present inventors. Also, its genome sequences were first determined by the present inventors. The present inventors have determined the full-length genome sequence of the bacteriophage PA1Φ. As a result, the genome of the bacteriophage PA1Φ was determined double-stranded DNA having 34,553 by of size and 50 open reading frames (ORF) (See FIG. 5). In addition, it was shown that the bacteriophage PA1Φ of the present invention had a remarkable killing ability against Pseudomonas aeruginosa and Staphylococcus aureus (See Table 1 and FIG. 2 to FIG. 4).

The bacteriophage PA1Φ has been deposited under the accession number of KCTC 11796BP. The present inventors have screened and selected a novel bacteriophage capable of killing Pseudomonas aeruginosa and Staphylococcus aureus. The resulting bacteriophage has been deposited at Korean Collection for Type Cultures (KCTC), Korea Research Institute of Bioscience and Biotechnology (KRIBB) on Oct. 26, 2010.

According to the present invention, the bacteriophage PA1Φ is characterized that it is capable of kill one or more bacteria strains selected from a group comprising Pseudomonas aeruginosa, Staphylococcus aureus, Staphylococcus epidermidis, Staphylococcus homonis, Shigella sonnei, Listeria monocytogenes and Streptococcus pneumonia. In Examples described herein, it is confirmed that the bacteriophage PA1Φ has remarkable killing abilities against bacteria described above (See Table 1 and FIG. 2 to FIG. 4).

According to the present invention, the bacteriophage PA1Φ can be applied for medical industry, food industry, biotechnology industry and the like. Without any problem related with resistance development of bacteria, this bacteriophage can kill Pseudomonas aeruginosa, Staphylococcus aureus, Staphylococcus epidermidis, Staphylococcus homonis, Shigella sonnei, Listeria monocytogenes and Streptococcus pneumonia effectively. Therefore, the bacteriophage PA1Φ may be used widely for anti-bacterial use against these bacteria.

According to the present invention, the bacteriophage PA1Φ can be used to remove biofilms generated by Pseudomonas aeruginosa, Staphylococcus aureus, Staphylococcus epidermidis or Staphylococcus homonis as well as to kill these bacteria highly as described above. Biofilms resist conventional antibiotic treatment and biofilm bacteria show much greater resistance to antibiotics than their free-living (planktonic) counterparts. Therefore, it is not expected to eradicate them sufficiently only by conventional antibiotic treatment. Preferably, the bacteriophage PA1Φ of the present invention can eliminate biofilm effectively. Hence, it is usefully applied to remove biofilm.

According to another embodiment, the present invention provides the pharmaceutical composition comprising the bacteriophage PA1Φ as an effective ingredient, which can be used to treat diseases that are caused by one or more bacteria selected from a group comprising Pseudomonas aeruginosa, Staphylococcus aureus, Staphylococcus epidermidis, Staphylococcus homonis, Shigella sonnei, Listeria monocytogenes and Streptococcus pneumonia.

Particularly, resistance development of bacteria to bacteriophage is much slower than that to general anti-bacterial agents and bacteriophage does not affect eukaryotic cells. Thus, the bacteriophage comprised in the composition of the present invention is useful to treat infectious diseases associated with animals including human.

As used in the present specification, term ‘treatment’ is intended to mean the prevention of diseases caused by Pseudomonas aeruginosa, Staphylococcus aureus, Staphylococcus epidermidis, Staphylococcus homonis, Shigella sonnei, Listeria monocytogenes, and Streptococcus pneumonia; the suppression of diseases caused by the bacteria described above; and the reduction of diseases caused by the bacteria described above.

As described above, the bacteriophage comprised in the composition of the present invention is capable of killing Pseudomonas aeruginosa, Staphylococcus aureus, Staphylococcus epidermidis, Staphylococcus homonis, Shigella sonnei, Listeria monocytogenes, and Streptococcus pneumonia, so that it may effectively treat various diseases caused by these bacteria even though becoming chronic after forming biofilm. The above diseases may include breast inflammations, skin inflammations, septicemia, purulent diseases, food poisoning, pneumonia, osteomyelitis, impetigo, bacteremia, endocarditis, colitis and the like. In order to acquire additional efficacies, the bacteriophage composition may comprise other effective ingredients that have already approved activities of antibiotics, especially against these bacteria.

The above-mentioned ingredients applicable in the present invention while having already approved activities of antibiotics, may include methicillin, oxacillin, vancomycin and the like, but not limited to, and besides, other antibiotics may be included.

The composition of the present invention may also comprise pharmaceutically acceptable carriers. The pharmaceutically acceptable carrier comprised in the present invention may be conventional kinds used during the formulation. It may include lactose, dextrose, sucrose, sorbitol, mannitol, starch, acacia gum, calcium phosphate, alginate, gelatin, calcium silicate, microcrystallic cellulose, polyvinylpyrolidone, cellulose, water, syrup, methylcellulose, methylhydroxybenzoate, propylhydroxybenzoate, talc, magnesium stearate, mineral oil and the like, but not limited to.

The pharmaceutical composition of the present invention may also comprise, if desired, additional ingredients including lubricants, humidifiers, sweeteners, flavors, emulsifiers, suspending agents, preservatives and the like.

The pharmaceutical composition of the present invention may be coated over lesion sites, or sprayed on lesion sites. Besides, it may be administered through oral or parenteral routes. The parenteral administration may be conducted by using intravenous, intraperitoneal, intramuscular, subcutaneous, local administration or the like.

However, the specific dose level that is coated, sprayed and administered for any particular patient will depend upon a variety of factors including the methods of formulation, modes of administration, the age, body weight, sex, severity of symptoms, diet, time of administration, route of administration, rate of excretion, sensitivity to reactions. Generally, the skilled physician may easily determine and prescribe the desired dose level of the drug composition to treat diseases effectively. Preferably, the pharmaceutical composition of the present invention may comprise about 1×10³ to 1×10¹⁰ PFU/mL of the bacteriophages.

The pharmaceutical composition of the present invention may be formulated by the procedures apparent to those skilled in this art. The amounts of active ingredients may be combined with the carrier or excipient material to produce a single dosage form or a several dosage form prepared within a container. In this case, the formulation may be in various forms, including solutions in oily or aqueous solvent, suspensions or emulsions as well as elixirs, powders, granules, tablets, or capsules. Additionally, dispersing agents or stabilizers can also be included.

According to another embodiment, the present invention provides an antibiotic comprising the bacteriophage PA1Φ as an effective ingredient. Preferably, the antibiotic can be an anti-bacterial agent for cosmetics or anti-bacterial agent for medicine.

In detail, Pseudomonas aeruginosa and Staphylococcus aureus killed effectively by the bacteriophage PA1Φ of the present invention, are noxious bacteria often found in cosmetics with Bacillus subtilis, Escherichia coli and the like. In general, the cosmetics are easily contaminated by bacteria and the like, because they may comprise oil or water as a main component and other combined substances, including glycerin or sorbitol as a carbon source of microbes, amino acid derivatives or proteins as a nitrogen source and the like. Besides, the cosmetics are even more susceptible to microbial contaminants, because of having a longer shelf-life than that of food. Therefore, in order to avoid de-coloring and de-flavoring evoked by microbial contaminations after storing for a long time, the cosmetics is required to supplement anti-bacterial agents additionally.

Recently, it is reported that synthetic antiseptic drugs such as parabenes most commonly used for cosmetics could be dangerous. Furthermore, the cosmetic comprising the synthetic antiseptic is recognized to be possibly dangerous. It is also published in the American Association of Dermatological Science that the synthetic antiseptic drugs should rank to the second causative agent of skin allergens. Currently, the artificial and synthetic antiseptics are concerned to threaten people' health, because of being utilized in children' products. Particularly, this may increase the time period of drug exposure and the accumulated amount of the drugs within a human body from childhood. Therefore, it is required deeply to develop new antiseptics from natural resources.

The bacteriophage PA1Φ of the present invention has advantages to remarkably kill Pseudomonas aeruginosa and, Staphylococcus aureus, compared to existing antibiotics and to effectively remove biofilms generated by these bacteria. In contrast to the existing antibiotics, the bacteriophage of the present invention is beneficial to exclude the possibility of resistance development of bacteria to bacteriophage, when being used for antibiotic. Therefore, the bacteriophage of the present invention can be provided for novel antibiotic that extends the life cycling of products, compared to existing antibiotic material. Most of antibiotic material is decreasingly utilized as the resistance of bacteria develops. But, the antibiotic comprising the bacteriophage PA1Φ as an effective ingredient could solve this problem related with the resistance development of bacteria basically. Indeed, the bacteriophage of the present invention is expected to extend the life cycling of antibiotic products likewise.

Therefore, the antibiotics comprising the bacteriophage PA1Φ capable of killing Pseudomonas aeruginosa and, Staphylococcus aureus, as an effective ingredient, is possibly applicable for better antibiotics outstanding in anti-bacterial effects, disinfectant effects and antiseptic effects. As used herein, term “antibiotic(s)” is intended to designate antiseptic agents, disinfecting agents and anti-bacterial agents entirely.

According to another embodiment, the present invention provides a disinfectant comprising the bacteriophage PA1Φ as an effective ingredient.

As described clearly above, the disinfectant that comprises the bacteriophage of the present invention to effectively kill Pseudomonas aeruginosa, Staphylococcus aureus, Staphylococcus epidermidis, Staphylococcus homonis, Shigella sonnei, Listeria monocytogenes, and Streptococcus pneumonia, may be applied for disinfectants usefully to prevent acquired infections in a hospital and human health area. Also, it may be useful for general disinfectants for life, disinfectants for food, cooking places and facilities, farm disinfectants for livestock industries.

Especially, the bacteriophage used for the disinfectants of the present invention is a very good sterilizer to remove noxious bacteria existing in general environment or medical devices, because it is environment-friendly due to being biodegradable and easily made to a liquid form. Besides, the cost of developing and producing bacteriophage is even lower than that of general anti-bacterial agents. Therefore, it is concluded that the bacteriophage could be efficacious especially for health industries.

### Advantageous Effect

As illustrated above, the method for killing Pseudomonas aeruginosa, Staphylococcus aureus, Staphylococcus epidermidis, Staphylococcus homonis, Shigella sonnei, Listeria monocytogenes, and Streptococcus pneumonia according to the present invention, is very important to treat and prevent diseases. In former times, in order to treat diseases caused by such a noxious bacterium and to eradicate their causative bacteria, chemical anti-bacterial agents have been administered to a human body, livestock or the like. Also, physico-chemical regimens have been used to lower the causative bacteria in food stuffs or environment. However, the abuse of anti-bacterial drugs becomes problematic to result in prevalence of drug-resistant bacteria, which may be a risk factor in respect of national health. Either, the physico-chemical regimen is disadvantageous to contaminate environment and to require high costs. In addition, it is shown that the bacteriophage PA1Φ of the present invention should remarkably remove biofilms generated by Pseudomonas aeruginosa, Staphylococcus aureus, Staphylococcus epidermidis and Staphylococcus homonis. Therefore, the bacteriophage PA1Φ that can kill these bacteria causing various diseases may is highly valuable to be used for food and environment-friendly anti-bacterial drug alternatives as well as health industries.

### DESCRIPTION OF DRAWINGS

The application of the preferred embodiments of the present invention is best understood with reference to the accompanying drawings, wherein:

FIG. 1 depicts the electron microscopic photograph of the bacteriophage PA1Φ according to the present invention.

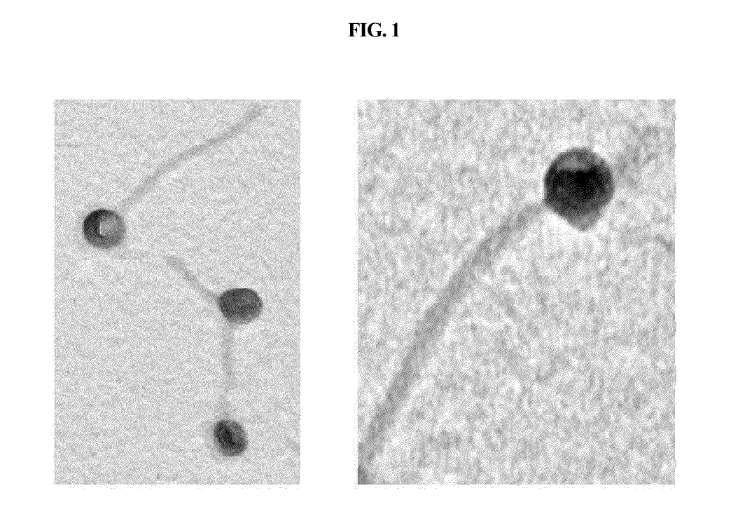

FIG. 2 depicts a graph of the killing ability of the bacteriophage PA1Φ against various suspended bacteria that shows the absorbance measured by using TTC.

FIG. 3 depicts a graph of the killing ability of the bacteriophage PA1Φ against various biofilm-forming bacteria species that shows the absorbance measured by using TTC.

FIG. 4 depicts the scanning electron microscopic photographs of bacterial biofilms after being formed for 24 hours in P. aeruginosa PA01 (A, E), S. aureus ATCC 25923 (B, F), S. epidermis KNUH-134 (C, G) and S. homonis KNUH-329 (D, H); and the scanning electron microscopic photographs of biofilms after treatment with the bacteriophage PA1Φ of the present invention for 3 hours respectively.

FIG. 5 depicts a schematic diagram of whole genomic mapping in the bacteriophage PA1Φ. The bacteriophage PA1Φ is composed of total 51 genes. Black arrows indicate structural genes; red arrows, genes associated with transcription and gene expression; and white arrows, theoretical genes.

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Production and Isolation of the bacteriophage PA1Φ

In order to isolate bacteriophages, waste water was collected near a livestock farm breeding pigs and cows and separated as described below. The waste water was centrifuged at 6,000 rpm for 10 minutes so as to discard large particles and solid stuffs. The resulting supernatant was collected. Then, 0.1 mL of P. aeruginosa (Pseudomonas aeruginosa PA01; ATCC 15692, 10⁸ CFU/mL) cultivated to a growth phase was inoculated into 4.5 mL of 10×LB and 4.5 mL of waste water obtained above was added to mix them. The resulting mixture was cultured overnight with a shaker at 37° C. Then, 200 μL of chloroform was added and further cultivated at 4° C. for 2 hours. Afterward, the resultant was centrifuged at 8,000 rpm for 10 minutes to collect supernatant. The supernatant was filtrated with a 0.45 μm-syringe filter.

In order to detect the presence of bacteriophages in the solution, various bacteria including Pseudomonas aeruginosa were spread onto solid LB media and then, dried. The solution was dropped onto the bacterial lawn, dried again and cultured overnight in an incubator at 37° C. (Spot test). Occurrence of cell lyses is determined by observing clear spots appearing on the solution drop.

In order to purify bacteriophages specific for Pseudomonas aeruginosa, culture solution of Pseudomonas aeruginosa was proliferated until reaching an exponential phase and mixed with the bacteriophage solution. The mixture was blended with 10 mL of liquid top LB agar containing 0.5% agar, then poured into LB agar plates and solidified. After that, the resulting plates were cultured in an incubator at 37° C. and the formation of plaque was observed (plaque assay). Then, a single plaque was picked and put into an exponential phase culture of the same bacteria. And then co-cultivation was performed for amplification of bacteriophages. After co-cultivation, plaque assay was performed again. This procedure was repeated to separate and purify the bacteriophage of the present invention. The resulting bacteriophage has been deposited at Korean Collection for Type Cultures (KCTC), Korea Research Institute of Bioscience and Biotechnology (KRIBB) (accession number KCTC 11796BP) and named with the “bacteriophage PA1Φ”. Bacteriophage stock solution was prepared using SM buffer solution (100 mM NaCl, 8.1 mM MgSO₄.H₂O, 50 mM Tris-HCl (pH 7.5), 0.01% gelatin) containing 7% DMSO and stored at 4° C.

### Example 2

### Host Specificity and Killing Ability of Bacteriophage PA1Φ Against Various Bacteria

The bacteriophage solution obtained in Example 1 was used. Various bacteria including Pseudomonas aeruginosa cultivated in nutrient media were shaking-cultivated for an hour and spread onto nutrient agar plates. Then, the solution containing the bacteriophage PA1Φ was dropped onto the bacterial lawn and incubated at 37° C. for 18 hours. Afterward, degrees of cell lysis were observed. The killing ability of bacteriophage PA1Φ against each bacterium is summarized below in Table 1.

TABLE 1
Host specificity of bacteriophage PA1Φ

| Bacteria | Pseudomonas aeruginosa |
|---|---|
| killed by | (ATCC 29853, ATCC 15692) |
| bacteriophage | Staphylococcus aureus |
| PA1Φ | (ATCC 25923, ATCC 29213) |
| | Staphylococcus epidermidis |
| | Clinically-isolated strain |
| | Staphylococcus hominis |
| | Clinically-isolated strain |
| | Coagulase-negative |
| | Staphylococcus aureus |
| | Clinically-isolated strain |
| | Listeria monocytogenes |
| Bacteria | Escherichia coli (ATCC 25922) |
| without being | Enterobacter aerogenes |
| killed by | Clinically-isolated strain |
| bacteriophage | Acinetobacter baumannii |
| PA1Φ | Clinically- isolated strain |
| | Serratia marcescens (ATCC |
| | 8100) |

As described above in Table 1, it is shown that bacteriophage PA1Φ could kill Pseudomonas aeruginosa, Staphylococcus aureus and Listeria monocytogenes. But, it did not kill Serratia marcescens, Enterobacter aerogenes, Acinetobacter baumannii and Escherichia coli. As a result, it is noted that bacteriophage PA1Φ had a broad host range, lysing Pseudomonas aeruginosa, Staphylococcus aureus and Listeria monocytogenes effectively.

### Example 3

### Morphological Observation of Bacteriophage PA1Φ by Electron Microscopy

In order to observe the bacteriophage PA1Φ morphologically, electron microscopy was conducted. About 10¹² PFU of purified bacteriophages were overlaid onto carbon-coated copper grids and negatively stained with 2% uranyl acetate. Electron microscope images of the bacteriophage PA1Φ were taken using a Phillips EM 300 transmission electron microscope.

As a consequence, the bacteriophage PA1Φ was observed to have an iscosahedral head and a long tail. Due to the morphological features, the bacteriophage PA1Φ has been classified to Siphoviridae family.

### Example 4

### Genetic Characteristics of the Bacteriophage PA1Φ

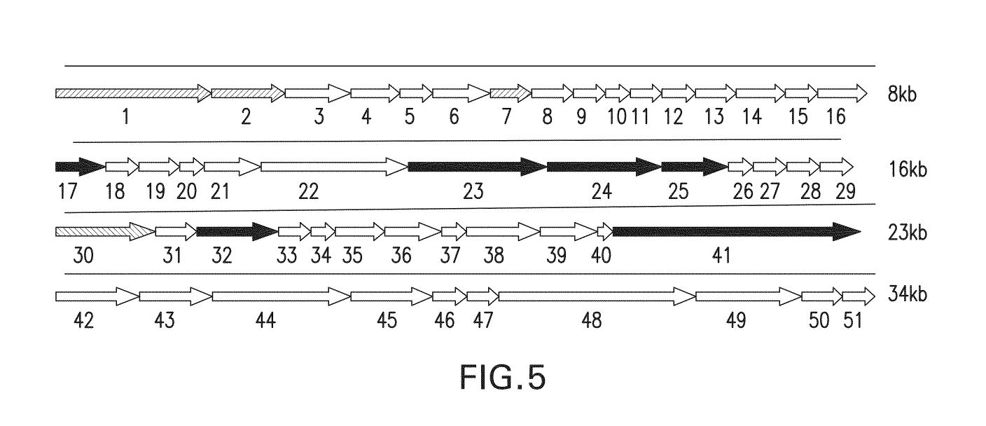

Genome of the bacteriophage PA1Φ was prepared by using a Lambda midikit (Quiagen). The resulting genome of the bacteriophage PA1Φ was delivered to Macrogen Co. Ltd. in order to determine the sequences of whole genome. In order to determine the total nucleotide sequences, shotgun cloning was performed, further cosmid libraries were constructed and analyzed by using an automatic sequence analyzer (ABI PRISM 377). From the sequencing data, open reading frames (ORF) were analyzed by using Sequin software version 9.5. As a result, it is found that the genome of the bacteriophage is double-stranded DNA having about 34.5 kbp of size (SEQ ID NO: 1) and is composed of 51 ORFs (See FIG. 5). Besides, the total nucleotide sequence was analyzed by using a BLAST program, NCBI, US. Consequently, it was determined to have 97% and 90% of homology respectively with MP29 and D3112 bacteriophages, both specific for Pseudomonas aeruginosa, and identified to belong to Siphoviridae family.

### Example 5

### Killing Effect Against Free-Living (Planktonic) Bacteria and Biofilm Removal Effect of the Bacteriophage PA1Φ

In order to examine the killing effect against planktonic bacteria and the biofilm removal effect of the bacteriophage PA1Φ, following bacteria were utilized: Staphylococcus aureus (ATCC 25923), Staphylococcus aureus WS-05, Staphylococcus aureus D43-a, Staphylococcus epidermidis KNUH-134, Staphylococcus epidermidis KNUH-174, Staphylococcus homonis KNUH-328, Staphylococcus homonis KNUH-329, Pseudomonas aeruginosa PA01 (ATCC 15692), Pseudomonas aeruginosa GFP.

Pseudomonas aeruginosa was cultured by using Trypton Soy broth (TSB). Besides, the other bacteria were cultivated by using TSB containing 0.25% glucose so as to collect planktonic bacteria. These bacteria solutions were adjusted to 5×10⁷ CFU/mL of concentration and 0.1 mL of aliquots were distributed onto a 96-well microplate. Then, 0.1 mL of 1×10¹¹ PFU/mL bacteriophage PA1Φ solution was added into each well. Afterward, the resulting plate was cultivated in an incubator at 37° C. for 6 hours. Then, 50 μL of 0.1% triphenyltetrazolium chloride (TTC) was added and incubated further in an incubator at 37° C. for an hour. After incubation, the optical density was measured at 600 nm of wavelength so as to calculate the number of living cells. Herein, the TTC, an index material of bacterial survival is used as a substrate to measure the absorbance of living bacteria.

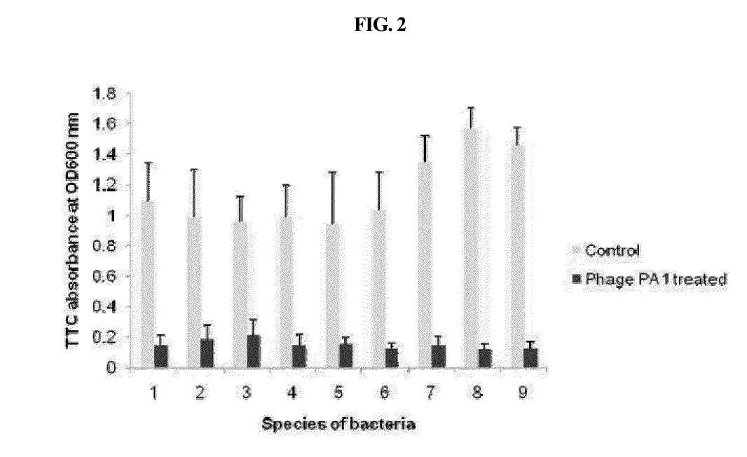

FIG. 2 depicts a graph of the killing ability of the bacteriophage PA1Φ against various planktonic bacteria that shows the absorbance measured by using TTC. ‘Control’ is a comparative group without adding of bacteriophages, and ‘Phage PA1Φ treated’ is an experimental group treated with bacteriophages. 1: Staphylococcus aureus ATCC 25923, 2: Staphylococcus aureus WS-05, 3: Staphylococcus aureus D43-a, 4: Staphylococcus epidermidis KNUH-134, 5: Staphylococcus epidermidis KNUH-174, 6: Staphylococcus homonis KNUH-328, 7: Staphylococcus homonis KNUH-329, 8: Pseudomonas aeruginosa PA01, 9: Pseudomonas aeruginosa GFP.

In FIG. 2, it is determined that all the bacteria used in the experiment could be killed to 80% or more by the bacteriophage PA1Φ of the present invention.

Furthermore, for the formation of bacterial biofilms, the bacteria solutions were adjusted to 5×10⁷ CFU/mL of concentration and 0.1 mL of aliquots were distributed onto a 96-well microplate. Then, the resulting plate was cultivated in an incubator at 37° C. for 24 hours and the culture broth was removed. The microplate was dried on a sterile place. After drying, 0.1 mL of the bacteriophage PA1Φ solution (1×10¹⁰ PFU) was added and treated for 3 hours. Fresh 0.1 ml of TSB and 50 μL of 0.1% TTC were added and reacted as described above. Then, the absorbance of the reaction mixture was measured.

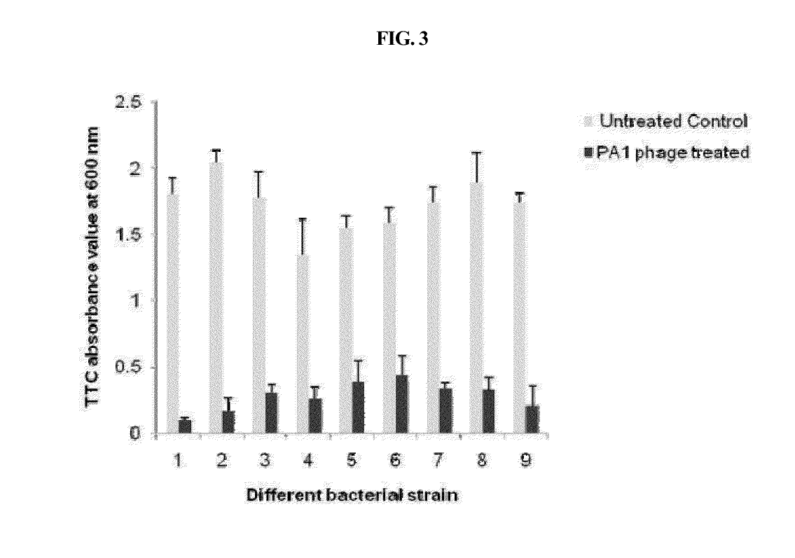

FIG. 3 depicts a graph of the killing ability of the bacteriophage PA1Φ against various biofilm-forming bacteria species that shows the absorbance measured by using TTC. ‘Control’ is a comparative group without adding of bacteriophages, and ‘Phage PA1Φ treated’ is an experimental group treated with bacteriophages. 1: Staphylococcus aureus ATCC 25923, 2: Staphylococcus aureus WS-05, 3: Staphylococcus aureus D43-a, 4: Staphylococcus epidermidis KNUH-134, 5: Staphylococcus epidermidis KNUH-174, 6: Staphylococcus homonis KNUH-328, 7: Staphylococcus homonis KNUH-329, 8: Pseudomonas aeruginosa PA01, 9: Pseudomonas aeruginosa GFP.

In FIG. 3, it is elucidated that all the biofilm-forming bacteria used in the experiment could be killed effectively. But, it is shown that S. hominis should be most resistant to survive in about 30% and Staphylococcus aureus should have the highest susceptibility (95% or more of the same).

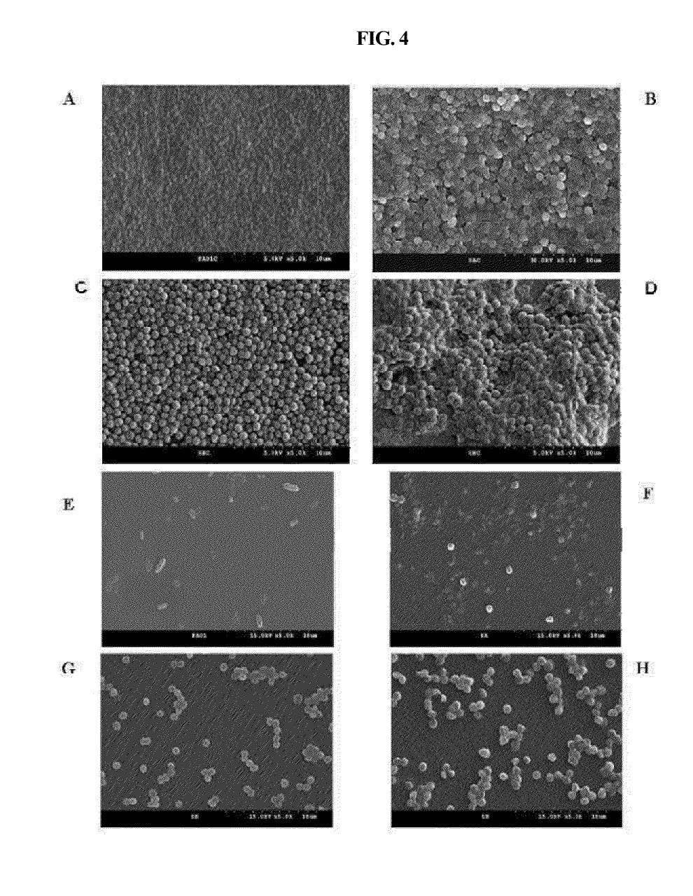

After performing the same procedure described above, the biofilm removal effect was examined under a scanning electron microscope. FIG. 4 depicts the scanning electron microscopic photographs of bacterial biofilms after being made for 24 hours (A-D) and the scanning electron microscopic photographs of the same biofilms after treating respectively for 3 hours with the bacteriophage PA1Φ of the present invention (E-H), in P. aeruginosa PA01 (A, E), S. aureus ATCC 25923 (B, F), S. epidermis KNUH-134 (C, G), and S. hominis KNUH-329 (D, H).

As illustrated in FIG. 4, the biofilm-forming bacteria were shown to make very close and dense bacterial colonies after 24 hours (A-D). But, it is observed that after treating the bacteriophage PA1Φ of the present invention for 3 hours, bacterial cells and biofilms should disappear mostly in Pseudomonas aeruginosa (E) and Pseudomonas aeruginosa (F). It is also noted that the biofilms should disappear and leave scattered bacteria in S. epidermidis and S. hominis.

Consequently, as illustrated above, it is confirmed that the bacteriophage PA1Φ according to the present invention is remarkable to kill Pseudomonas aeruginosa, Staphylococcus aureus, Staphylococcus epidermidis, Staphylococcus homonis, Shigella sonnei, Listeria monocytogenes and Streptococcus pneumonia. Furthermore, the bacteriophage PA1Φ can remove effectively the biofilms generated by Pseudomonas aeruginosa, Staphylococcus aureus, Staphylococcus epidermidis and Staphylococcus homonis.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An isolated bacteriophage PA1Φ belonging to the family Siphoviridae, wherein it is deposited under the accession number KCTC 11796BP.

2-4. (canceled)

5. A pharmaceutical composition comprising the bacteriophage PA1Φ of claim 1 as an effective ingredient.

6. An antibiotic comprising the bacteriophage PA1Φ of claim 1 as an effective ingredient.

7. A disinfectant comprising the bacteriophage PA1Φ of claim 1 as an effective ingredient.

8. A composition comprising an amount of the bacteriophage PA1Φ of claim 1 effective for the removal of biofilm generated by one or more bacteria selected from the group consisting of Pseudomonas aeruginosa, Staphylococcus aureus, Staphylococcus epidermidis or Staphylococcus homonis.

9. (canceled)


SEQUENCE LISTING (1 sequence)

SEQ ID NO: 1 - DNA, 34553 bp, Bacteriophage PA1
tccgaaagctggcagaaagggaaggctggaagaagcagaagcactccgggcgcggtggtggatatgagtaccacgtttcggccctgccgaaagaaacccgcgccgcgctgctgaacgccgccctgggcgaggtggccaccaaggtggtccgccaggagacgcaactggccctggtcgaaaccaaccgccagcagctggtcgccgatgcccgccagggggtgctgcacgccctggacctgatgatggcccgcaccggctacagccggaaacgctccatcaccctgatgctggacatggcgcgcctcggccaggtcgagccgcaactgctcgccatgctcaagatggcccgcgatcctcgcggccgcccgagcgcggatggcctgccgagcgtgcgcagcctggagcgcttcctggaccaagccgagcgtggcgccctggtgccgaaggtccgccgcccggacatgagcgttcccgactgggcgccggcattcatgacgatctaccaggggccggagaagcgcagcgcccgcgctgcccatgcactgctggagaagcactggcaggggcaaatgcccagcctggaccaggtctatgcgttcctgcgcaaggtcggcaacgtcagccgcgaggtcgggcgcatgggtgagcatgaaatcaaggcgctgcgcccgttcattcgccgcgacttcaccaagctgctcccgaccgacgtctattcatgcgacggccacacgttcgacgccgaggtccagcacccgatgcacggccggcccttccggccggaaatcaccaccatcatcgacatccgtacccgtcgaattccgggatggtccaccggcttggccgagtcggccctggtggtggtcgatgccctgcgtgacgcctgcaccaagggcggcattccggccatcttctacgtggacaacggctcgggctacatcaaccacatgatgcgcgacgaggcggtcggccttatgggccgcctgggcatcgatatgaagaacagcctgccctacaacagccaggcgcggggcgtgattgagcgcgtccaccagagcctgtggattcgggccgccaaggaactgcccggctacatcggcgccgacatggaccgccaggccaagctggccaccttcaagctgactcgccgggccattgccaagggcggcaccatgccgctgatgtcttgggaatcttttgtcgcgttctgcgagcaacagattgccgagtacaacgaccggccgcacagcagcttgccgcgcatcgttgacccgaacaccggccgtcgccggcacatgacccccaacgaagcgtgggcgctccacgaagccgatggcttccgcccgatgcgggtcaccgacgacgaggcccggccgctgttccggccccaggtgctgcgcaccgtccgccgctgcgaactggagttcatcggcaaccgctacttcgcccgcgagctggaggaattccacggcgaccaggtggccgtgggctacgacatccacgacgccagcaaggtgtgggtctacgacggcgagggccgcttcctttgcaccgcagagctgaacggcaacagccgcgactacatgccggcttcgtatgtcgagcgtgcccgcgagaaacgcgcagaggcccgcgagaagcgcgccctggctcacctcgacgagattcgcgccgagcgtgacggcgggtatgccctggaaatggatgcgccgctgtccatccccggcctcggcacgatcacccctgagcaactccggtcgcgcagcgccgcgaccctcgaaatgcaggccgagcggatcgacgaaccgcgcccggccgcagccaccgcccaagccaccaccgcccaggtcttcaccctgccgaccgctcccgcccagcgctaccggcagtggtgcgagctggccgagcggcagcgctccggccaacccatcgagccggacgccgcccagtggttcgaggtttaccccaaatccaaagaattcgccgcccagcagcggcaagcatgaaaggagctgtattcatgaccaccccgaaaaccacccaactggccagcggcatggccgacatcgccaacatcgccctttgcgatatcgccctggagaaagcgctgtcgcgtacctccaccttgcccggcctggtctgcttctacggcccgtccggcttcggcaaatccgtttcggccgcctgggtcgccaatcgccgccgcgcctactacgtccaggccaaaagcgtctggacccgcaagcacacgctgaagtcgatcctgggtgaaatgggcatcaagccggccgggaccatcccggaaatggccgaccagatcgccgaggaactggccgccagcggccgcccgctgatcatcgacgaaatggaccacctggtcgccgctggccaggtcgagctgattcgtgatctgtacgagtccagccaagcctccatcttgctgatcggcgaggaaatgctgccgaccaagctcaagaagtacgaacgcttccatggccgcgttctcagctgggttccggcccagccagtgtccctggaggacgcccgcaacctggcgccggtctacagccccggagtggctatcgctgacgatttgctcgcgcacctggtgaagaagtccctgggctctgtccgtcgcgtcgcggtgaacctggagcagttggccgaggccgccaccgttcagggccggcgcgagctggaactggccgacctccagcgcctgaacctggagctgtataccggcgcggccccgagcccgaggacttcgaagtgagcctcggcaagaacccggcccacctgtctatggtcgggggcaagagcccccgccagcagatgtgggaagtcatccgggccaaccgcgaagagttcaccgtctaccgcgtggcgcgccgctccaaccagcacgacaagaccgtcgagaagtatgtcgcctgcctgcgcctgggcggctacgtcgaggcgatccgtggattcaagcgcggcgaagaagtcgttttccaactggtccgcgacaacggcgtcgaggcaccgaacctgaacgccgatggcaagccatcccagcagggctacaccaccgaggcggtctggcggacgttgcgaatcctcggcccatcgaccccggaacagatcgccgcatcggtggcggcctcgggcacgcccgtgtcgcccagcaccgttcagcgctacttcatcgacctccaaaacgccggatacctgacccgcaacggccgccactacgccctcaagccgggccgctacaccggtccacggccgcccatcgtccagcgcgagacgcgccgccaggtctacgacccgaacctggaccaggtcatgtggagttcgcacggcgagtaccagcacaaccggagccgttccaagaaatcagaacaggaggtgccgccatgcgccgagcactgatccccatcggcatcttcctggccctcggcctgctgctggtcctggccggtgatgccctgatgctcggccgccgcctcattgcctggcaatgggggtgctaatggaccgcgcaatcgatctgtcggcctggggcgagcgcccgcccgtcttcgtccaactgctggccgctgaggtggcccgcagcagccagacgaaagccggcgaggcaatcggcatgagccgttcgaccgtcagcaccatcctcgccaaccgctacccgtcgccctcgacgatccgcgtcgagcgtcgtgtcctggccgcgctgagccgcattgagtgcccggccctgggcgaggcggtgacctcggtcgagtgtagcgagtacctccagcggccggcgccgctgaacaaccccgtcgcgatgcgctgctggaaagcgtgccgcgcctgtccacgcaacccgcataccgcccccatgaaacgagaggaacaaggccatgagaaccgcattgcccttgaaagtcttgacgcctgacttggccctgagcctgcgcaccttcaacgatgccgcccggttgctccagcgcatgggggttcgccttcatcgcctggagccgacagaggggcgcgtgaccatcgccgcagatgacgcccgccagctcctggagaaggggctcctgctgggtttccagcgcgaggcctcggccggcagcacccgttacatcacccgcttccagggcatcaccctggcctggagcgaaccgatcagctaccgcgacttcgccggcagcaaacccgtaattcactgaacaggagaacgccaacatggcatcgaagaaacgtctgaaatccgctgccgccgtctacgtcccgcagacccgcgagcaggtcatcagcgatatcaagaacatcggcgacctccagcgcgagctggcccgtctggaaaccgcaatgaacgatgaaatcggccagatcaccgagcgctattcggagccggccgaagacctgaagaagcgcctggcggtcctccagggcggagtccagtcctggtgcgaggccaaccgtgccgagctgaccgacaacaacaaggtcaagtacgcgaacctcacgaccggcgaggtccagtggcgcatccgtcccccgtccgtgactgtgcgcggcgccgatgcggtcctggagctactgcgcagcaaggggctaatccgcttcatccgcaccaaggaagaggtgaacaaggaagcgatcctcaacgaacccgaggccgtccaggggctgccggggctgaccctgaacacgggcatcgaagacttcgccatcgtgcctttcgaagcggaggtgcagtgacatggccgaggaaatcagcatcgacaccatcatgtcgcaggcccaggtattcgccagtgcttgggcgctggtcgggggcacattcgacgacggccacgccatcgagaacgctgaggaagccaaggctgaactgcgcgaaatgctggaggacttctgttcgaacactgacctcctgcgcgtggctgagctgctcacctcttggcaccagaacgggatgggcaagattgatcaggcgctgaatgcgccggatacggccgaggttcggattggctcggccaggctcactggttcccaagccatcggcttccgaatcggcctacgagtggcccgccagtggcttggcacactcccgctatcgctcaccaaacaggaggtgtgacatggcccgcaaccgcgcgcaacagctgtgcatcgtcaccttggactatcagcgcttcctgttaccccaggctgatgcactcaagctgatagacatcatgagtcgagccgcagaggttcaggccgactacgcctctggagctgggttcaagtacaccgtcggcgaagtgccggaagtcgagttgacgatagtacgccccagtcaattggtcatgccgcaggccgagccggccccagctacaccacgcgctcgccggaagtctccggcccaggtcacgcacgatgccattcggctgctggaggggctctgatcatgaccaagacgttcgccatgtgccgcatcgacggcctgatcgagctgcgggaggaacacccaggcgagggctacttcgcccttgccgtgggcgacttggccagcgtgcgggcggcggtctttgcaaccgctgagccgcaccaggtcggcaagaaagtcgcccggcgcgtgccgggtgtgagccccgacgccaccgaccgcgaaaacctgggctccatcgcccggtacatccagaccctgggccagcaggatcggccgggcttccgtgcgctgggggtgtgaaatgcagcagtccaaccccttcaatcaccccggacagagctacggcgccgtagacgtcgatagccgactccgcgccgttgccggcttcgacctggagcaatgccgcgccgcgctcgcggtcaccggcctccagaagatcgtcgagcagaaaattcgcacccgcatccgccagctggaaaagcaggcatccgcacagaaggaggcataaccatggccgtatacaccatcaccctcagcgacaccgagcgcggaatagatttctccatgcaaggcccgccgctgcacgactccgaagcatcgaaggtcgcctatgcccttatgcaagcgacgatgtccctcggccaggaactcgcaaagcagaacggagctggtaacggcgtttcctgcgcctgcgacgagtgcctggcgcgccgcgctcgcggcgaagaaccgcagcaggaaatccactacaccaaggccaagaaccgcaccgtccattgagcgaaaccgccccggccttgccgggacggtctgccggacgtggtggtccggtactgatgagcagccacccatgacgaacgaaaccaagacagaccgccagcggcgcctggcgcgggaacgccaacgggcgaagcgcgagcgcgatgccctgcgtcgcgctgcgctgggcggccgccgcttcaacatggacatgtaccagggaacggctgatgcactcgatctgatctgcgcggccggtggcttcgccgagccggccgaggcggtcaccttgctcctacacaacgttgccgaaattgcagagcgtgacgcgtcacgttttgccgagttgatccaaaagagaagccatccagggaggaccaagcgatgagcctacgcgccgtcaacctcgcaaaaatccacatcgccaaggcccagctgggcatggacgatgacacctatcgcgcattgctggctcgcgtcgcgggcgtgcgctcggccaaggacttagggccgcgccagatcgaccacgtactggtcgaactccagcgcctgggctggaaaccgaagagcaaccggcagggccgggcgacgccaaaagtgccgcaaaaccggcaaaccgtgctgcgcaaaatcaccgcgctcctggccagcgcccatcgcccctggagctacgccgaccacatggcccggcgcatgttccaggtcgagcgggtcgagtggctggacgacagccagctctaccggctgatgcaggcgcttatcatcgataggagccgccatggccaggtctgaggtggatcttcgggaggtccaggacatgctgccggataccgtccgcgacatggccggacgcataggactgccggccaccctggtggtggtcgagcagctcggcggaacgtcctggcggatagccgagggccgggcgcggaggggcgaagcgcgccgggctgcgctggccgagctggtgggcagcgacatcgaggagcagctccacacgcactatcggggcgaagaaatttacgtggcccgctgccacaaggcgctggtacggtggcgcgatctggagatcgtcgagcgcttcgaacagggcttgcgtgatgggcaaaccgcccgtagcctgctcagcgatctagcccgccagtacaacctgtccgaccgctggatatgggagattgtcaaccggccgagcgagccggcaccgcagcaatccaccctgttccactaagccggggcgcaacgccccggccggcgtctccgcgccgatcctatctcagccgttgaaccccttccgctaatcccgcgtcgcactcgccgccacgatggcggcatgagcacatctagcccctcaacgtctctacgcagcccccgcgactacgccgccgccatcctggccgagcccagccgcgagcgtcgtaacgctctgctggcagcctgcccggccaactggcagccgcttgttcgggcgcacgtcgaggatgccttcgcgaaggtcaaggcgtatcgccagatgatggacaaccgcgccgagtcgatccggcgcggcccgcctcctgctccccgcgtcaccgacaccgatttccgcatatccaactacaccaagtccgccccggaggtaggcaatgcgcacctatccgcaattcgggcagcgctcgcaacggaagcaccaaatgcctgatcccgcatccacctcggccggcagcgccgcgctgctgaaaatgttcggcatccacataagcgcgggcgccctggctgccgccctgggcttccttgtcctgtggccccgaacaatgaaagaggggttcgcccggctgttctgcaccatcgtcgcgtccagcgtcttcggcccaatcctggtggtttacctgcactccaaacgccccgagttgttcgagtcggcccatgtggtggccgggctctaccagctggagccagcggtcggcctgctgttcgtttccgctccgctcctggtgattgccggtctgcctgcctggtggctgatcggtgcggccctgcgcctgtttgagcgggacggcgattcatggctgggcgcgttcgcccagtgggtaaaacgcaaactggagaacaactgatggcccttcaacctcgcggcatccgcaacaacaaccccggcaacatcgtttggtcggcacgcaacaactggcagggccagctcccgcacaacccgaagatcgagccccgattcgcccgcttcgacaccgcgcataacggcatccgcgccctggcaaagctgctgctgaactatcgcaaggtctacggcctgcgcaccgtcgaatcgttgatcgcacgctgggcgccgtccaacgagaacgacacccgcgcatatgccacggccgtggcccgagccatgggggttccgccgcaggccggcctgcacatggaccaggacaccctggccgccctggtgaccgcgatcattcggcacgaaaacgggcagcagccctacagcgccgagcagatcgcccaggctgtgcgggaggtgctgtgatgcagcgccccagcggaatcagcctcagcgatctgttcgcgatctgccgtgaagacccggccaaccgatggctctggatacgcctctatctccgcgacctgctggcccgcgtcgtgattctggtcttcatggcaattggtgccgcaggcctcgcctatggcctgggcggggcgttcgcctacggcttcatgcagaccgttgcgtcctaccaggtccagctcagcgtcgagaaatcgccatgacctggcgcgtcggcctggttgtctttgtgctcctggtgatggtctggacggccggctggtggggcggtcgcgaggccggtctggccgatgggcgcgccgcctgcgctgacgcacagacccgcgcatatcgcgacgtcctggagcaatcggcggcacaactgaagacggtccaggacaccagcgcggctcttttccagcgcctggcccagcgggccgacagcgaccaacaaactactcgggagcttcgccatgccctggccgaaaccgctgctgatcgcgctgcctgccgctttcctgctggcgtcatgcagcagctcgaaaccgcccgtcaacgtgccgcccaggccactaccagcggccttggctcaaccgtgccagaccccggtggcggtgactgatgacagccccgatgccgccgcaattgcccttaaacaactctacgaccaatacggcgcttgcgccggcctgcactgggacaccgtgcggcaccttcaaaaggactgatccgatgaccgaaaagaaagcctctcctgagttcgaactgctgcaacgcatcgacggccgcctggagcgcttcgaagaccgattcccgcagatcgaacgcaaggccgtgctgtacggctcggcggccggcgcgctggcgggtggcctggttgcctgcggcctgctcgcggcgcgtatcaagctcggtatctgaggtagtcgatggcgcacccgaaggaaacccgcgacgccctgcgccgcgcctacgtcctcgaccgccagtccctggaggtcgcggccgccatgttcggcgtctcctacggcaccgcccgccgctggaaacagcaggcggaagccgagggggacgactgggacaaggcacaatcggcgcagttgctggccggtggtgggctggaggacgtggcgcgccaggtgctggccggcctggtgacccagttccaggccaccatggaggcgatccaggtggacagcgcgatcacgccagcggtcaaggtgcagatgctcgccagcctggccgacgcctacaacaagtcgatcagcgcatcgaagcgggtactaccggaaacctccagcctggccaccgcaatggaagtcatccagcgcctcgcggccttcatccgtgaacagttcccgcagcacgtccaggccttcgccgagattctggagccgttcggggaagtaatcgctaaggggctgaaatgaacaccgaggaaaaagagttcctacgggaactatccgccatggcgcagcagctgcgccgcgacatcgaggcgcaacaggtcggcctcgatagctccccggaagctcgggctgagcggcgccgtcgcgtactggtagatcgcgatttcgagttcttcgcgtatacctacttcccgcaccacatccggccgcctgcctcgttgttccatgcgcatttcttcaagcgctttccccagctcatcagcacctccagcggcctcaaggaatggtgggtagctccgcgtggcgaggcgaagtcctcactgctgaccaaggtcgggccgtgctatgtcgttgtccaggggctgctccagcgaccggagattcgggccgaactgggcatgaccggcccggcgccgtacttcgtcgactacatcacgctcctaggcgccgaaacgcgcttgcccaccaagctcctggaagtagtcaagaccgagctactggtaaacgcctccctgtccctggacttccccgaagtctgcggcaagggaagcgtctggaagatcggcgagtttgtttccctctccggggtcaagctggaggccttcggcgctgaacaggcgatccggggcacattccacggcgcgagccgtcccaagctgctcctgggcgatgacctgatcaccgacaaagaggccaagtccccgaccgagcgcaacaaccgctgggactggctggaaaaggctatcgactacctcggcccaccggatggctccgtcaaatatctaggcgtgggcacagtgctgaataaggacgatccgatcagccgagccaaacgcacggtcgggcacctggtccaccacttccgcgctatcgagacgttccccgcccacatggacctatgggcacattgcgaagaggtgatgctcaacgacgacaagcctgtgatggagcaatacgccgagcgcggtagcgtcgcgcccgatagcgctctgccgtcgtttcagttctaccaggataaccgcgagcagatggaactgggggccgtcaccagttggccaggtgtccggtcgctctactggctgatgcgtcagcgggcgaagaacaaagccgcgttcgcgaccgagctgcaaggcgacccgcgatccgatgaagacaagacgtttaccaacccgcgcttttgggtcatgcgctccggccgttggcagatgtttggcgcctgcgacccctctgtcggggcgagcgcacagtccgacccgtcggcaatcatcgtggggggctgggacaccgagaaacaggtactcaacgtcatagaggcggccatcaagcggcgcgtcccctcgaaactggaatcggacttgatcaaggcgcaaagggagtaccagatgcgcgctatcggtttcgagaacaacggggcattcgaaatccagcggcagaacatcgccaaggctgcgctgatgcagcgcgtagcgctccctctggtcggtgttaccagcatggcggaccagtccgttcgaatcgatgccatggagccgttcatcaacgacgcatttgcgccacggattctgttttcacctggattggtcgccctgctcgacgagctggacagctggccagaaccgcagaccgggcaccactacgacggcctttgcgccctgtcgatcctctggatgatcgctagcacccgcgctggttcctatgaattcaccccggtgcctggccgtcgcagctctgcggattccagtgagttcaaagactcttttgacataggtggccgcatgggcggcgactggtaggagacgcaacgatggccaccatcgtggatatttacggcaaccccctgcgaacccagcagctgcgcaagcagcagaccgcgcacctgacgggactggccaaagagttcgcaaaccacccggccaaggggctgaccccagccaagttggctcgcatcttgatcgaggccgagcagggtcaactccaggctcaggccgagctgttcatggacatggaggaacgcgacgcccacctattcgcagaaatgagcaagcgcaagcgcgctgtcctcggcctggactggaccatcgagccgccacgcaacgcctcggccgcagagaaggccgacgcggagtatctccacgagctgctgctcgacctggagggcattgaagacctcatgctcgattgcatggatggcgtcggccacggctatagcgctatcgagctggactggtccctccagggacgggagtggttgccgcaggccttcgaccaccggccgcaaagctggtttcaactgaacccggacgaccaggacgagctgcgcttgcgcgataacagcatcgcgggcgaggtactccagccgttcggctggatcatgcacaagccgcgttcgcgctcgggatacgtggcgcgtagcgggctgttccgcgtgctggcctggccgtacctgttcaagcactactccacggccgacctggcggaaatgctcgaaatctacggtctgccgatccggctcgggaagtacccgcccggcacgcccgacgaagagaaggtgaccctgctgcgggccgtgaccggcctcggccatgccgcagcaggcatcatccccgagagtatgtccatcgagttccaggaagcgtcgaaaggctcggccgagccgttcatggccatgatgcgctggtgcgatgactcaatgtcgaaggccatcctgggcggcacgctcaccagccagaccagcgagtcgggcggtggtgcctatgccctggggcaggtccataacgaggtgcgccatgaccttctggcggcggatgcccgacagctcgccgccacgctgagccgagacctactctggcccctcctggtcctcaaccgctccggcaacctcgacgcacgccgcgccccccgcctggtgttcgacctcaaggaccgggccgacctggccgccatggcaacgtcattaccgcccctggtcaagttgggcgtccaggttccggtcaactgggtccaggagcaactgggaatcccgctgccggccaagggcgaggcagtcctggtcgatcaggccggcgcaggcatcgcccaactgagccggcgccctggtcctcgcgtcgctgcgctggcccaggtgattggaccacgctaccgcgatcaggaagcgctggaccaggtgctggccagcctgccagcccaggacatgcaaaaccaggccgatagcctggtcgcgccgctcctggatgtgatcagccgtggaggtagcgaggcagagctgctcggcgcgctggccgaggcattcccggatatggacgatagcgccctggcggatgccctccatcggttgctgttcgtggccgacacctggggccggctcaatggcacgttggatcggatcgactgatggcaacgccaaccgaggccgatctgcgggccatcttcgccatgcggccggaggccgccattgagtacctggagcgcaagggattcgccattacctggaactggcacgatgttgacgcggccacccatgcccgagcgctgacggtggccaaggcggcacgcctggacgtgctccaggacatccgcgacgccctggtcgataacctggagcgtggcggaacgctgcgcgacttccagcgcaacctgcggccgatcctggaggccaagggctggtgggggcgtcagatagtggttgcgccggacggcggggccgaggtcgcccagctcggcagcccgcgccggctggaaacgatctaccagaccaacgtgcagtcggcctacatggccgggcgctatgccgccgcatacgaggccagggaaactcacccttactggatgtatgtggccgtcatggacagcgtcacccggcccagccatgcggcgctacatggcaaggtgttccgctgggacgacccgatctggcagcacatcatgccgccgaatggctacaactgccggtgccgaatcgttccgttgacggcggccgctgtgcgtcgccgtggtctgacggtcgaatccagcgtcggcaagaccggccaggtgaccgtcgagacgggcgtagacaagcggacgggggagattcgggagcagaccttgaccaccctggagacgaccgaccgggccggtcggaagatccaatttcgccccgatgccgggttcgacggcagcccgatacagagcgccctgatggaccaggtgctgtacaacaaggccgagcgcaccctgggagcgcctgccgccctcggcgaggtccaggacgtgctcctggacccggtacgccagcgcgcttggcaagcgtttgtggaccgctccacgtcgccccagggacagacgatgtcggtcggcgttctcgatccgaccgacatcacctatgcggctgcccagggtgcccagctccaggctggcgtggtatcggccagcgacaccgtgatccgtaacagcccggtcgctcgcgagcagctggcgaacctgccgcagcgcctggctcagcccgccatggtgctgtgggagcgtggcagcgaatccctggtctatgtcgtccaggacggcgactctaccctggcggttcgtctgcgcggtggcgtctatgggccgggccaaatggagaacatcagccaggttacagaggtgacgatggaaagtatcgacgacgggctcgccctgggccgttacaggagggttcgctaatggccaatcgcatcgagctggaactggtggaccgcgaggtccaggagcgcctggcggcgctctacgcggcagtaaccgacactctgccgctgatgcgcggcatcgctgctgagctgctggccgagacggagtttgcattcatggacgaggggccgggatggccccagttgagccccgttaccgttgcagcgcgagcggcgaaggggcgcggcgcgcatccgattctccaggtcaccaacgccctggcgcgctcgatcacaacccgcgccgaccgtgaccaggcgcagatcggctccaatctgacctacgcagctatccagcagctgggcggccaggctggacgaggccgtaaggtgacaatcccagcgcgcccgtatctgccggtcctcagaagcggccagctcaagccaagcgcccgcgatgcggtcctggacctcctcctggctgttctgtcccagggtcgctagagggaatgcattcggataagcggcacattgtgcctattgtgcattaggcacaatgtgcctaatatggcgtcatgccagccacaacggcgaggcgccaagaaggatcgaagccatgagcacccaacatacttacgaagaaatcgctgaagacttccgtctctggggcgagtacatggaccccaacgcagaaatgaccgaagaggaattccaggccctctccaccgatgagaaggtcgctatgcaggtcgaagcatttggcatggaaggtgagcaatgagcaacacgatttcagatcgcatcgtcgcgcgctcggttattgatgcggctcgattcatccagtcgtgggaagatgcagaccccgacagcctgactgaaagccaggttctggccgccgctggtttcgccgccaggctccatgaggggctccaggctaccgtcctgcaacgactggtggacgagtccaatcatgaagagtatcgcgagttcaaggcatgggaagaggcgctgctcaacgcagatgggcgggtcgcgagcagcccgtttgccgattggggatggtggtatcgcatcgccaacgtgatgctggccactgcctcgcagaacttaggcgtcacctggggaagccgcgtccatgggcgtttaatggctatttttcaggacaagttcaagcagcggtatgaggaacaggcatgagccgaccaacagtcgttacggtgacggaaacccccaggaatccgggaagctacgaggtcaacgtagagcgggatggcaaaatggtcgttggccgggcccgcgcgggaagcgatcccggcgcagctgcggcgaaggccatgcagatggccatggagtgggggagcccgaactacgtcattctcggcagcaacaaggttcttgcgttcataccggagcaactgcgggtgaaaatgtgaaacctgacgcctccagccacaatccagacccgcgctacctgcgcgggctgctcaagaaagccggtatcagccagcggcgcgcagccgagctgctcggcctcagtgacagggtgatgcgctattacctgagcgaggatgtcaaagagggctaccgtcccgcgccgtataccgtccagttcgccctggagtgcctggcgagcgacccaccatctgcgtgatcacctgatccgcccgcaaacgcgctacacgcgccgaaacggggttagccgctacctcgcatcagagtcggtgcgttaaccccgttagaaccccgttagaaatcgttccatcgccatccgcgtgccagggcttagccagaagacggcgccggacggtttccgcagtcgttgaaccccttcacgtaaccgccgcgctcgaccgtcgccaccattggcggcatgaagaagaaccgcctacacgttgccatcgccgcctgctcgttccagctccccaagctggaggacggcagcgcctggattcaagttacgcctgccggtgagttctggcccatggatgggcgccctatggatgtgccgggctggcggatcgatgccgccagtgccgccgcagtgatcgagcgcgcacggtcgcgcaagactccgcccgtcctggactacgagcaccagaccctaaagaaagagcagaacggccagcccgcgcccgctgccggccgtttcctggacttcgaatggcgcgaaggctccggcctgtgggggcgtgtcgaatacaccgcccgcgccgcgaagctgatcgaggacggcgagtacctctacttcagccccgtcttcagctacgccccggacggcacggtcctctccatcctcatgggcgcaatgacaaatgaccccgccatcgacggtctggagcctctcgcacgccgagcggccgcgacctttggcctctacaaccccgacgaggaaacccctgtggatgaactcctgaaagccatcatcgcggccctgtcgctgaaagaaggcacgaccgaggcagaggccattgcggccctgaccgccctgaagccggccctggacgcccaagcgaccaacctggccaagctgcgcgaaaccctcggcctggcccaggacgcggacgtcgagcagatcgccgccgccaccgcccagctgaaggtcgccgcccccggtaaccccgacccggcgaagtgggcgcccgttgaagctgtcacccagctccagggccaggtcgcggcgctgacctctcgcctcaacggcggcgagctggacggcctgatcaacagcgctatccaggagggccgactcattccgtccatggagccctgggcgcgtgaatacggcgccaaggacttggccggcttgaagagctacctgggccaggccaagcccatcgccgccctgacccaacagcagagcgccgggcgtacctcggtgcctacctcggttgaccagctggacgaggccgccctcgcggtttgctcggccatgcagatcaagcccgaggattacctcaagaccctgaaaggccagtaaggaggcgctatgaccgccctgaccaccgaccgcaacaccccgctccaggacgccgaggtcatcggcgtgccggtagcggccaacgtccaggtcttcgccggcgccatcgtcgtggcgaacgctaccgggttcgccgtgggcggcagcaccgccaccggcctgacctacctgggccgcgctgaggaatacgtggacaaccgcaatggcgcggatggcgccaaggtcgtccgtgtgcgccgcctgaacgccttcaagtgggcgaacgacggcagcgtcacccaggcgcacctgatgaaacccgcctacatcgtggacgaccagaccgtcgccgccacggacggcaccgaaacccgctccccggccggccgcatcatcggcgtcgaaccggacggcgtgtgggtggaataacaggctcaccaacggagaaccggacacatgctgatcaacaagcagagtctcaacgcggcattcgtcgcgatcaaaaccatcttcaacaacgccttcgcggcggcccccaccacctggcagaagatcgccatggaagtgccgagcaacaccagcagcaacgattacaagtggttgagcacctttccgaagatgcgccgctggatcggcgcgaaggtggtcaagaacctgaaagcctacaagtacgttgtcgagaacgaggacttcgaggccaccgtcgaggtggaccgcaacgacatcgaggacgaccaaatcggcatctactcgccccaggcgaagatggccggttactcggcggctcagctcccggacgagctggtctatgaagcggtcaacggcgccttcaccaagccctgtttcgacggccagtatttcatcgacacggatcaccctgtcggtgatgcctcggtgagcaacaagggcaccgctccgctttccaacgccagccaggcggcggcgaaggcaggttatggcgctgctcgcactgcgatgaagaagttcaaagacgaggaaggacgttccctcaacgtctcccccaacgtgctcctggtcggcccggcgctggaagacgtggcgaagatgctgctcaccaacccgaagctcgcggacaacaccccgaacccctacgtcggcactgctgagctggtagtggacgggcgtatcgagtccgacaccgcctggttcctgctggacaccaccaagccggtaaaaccgttcatcttccagcccaggaaacagccggaattcgtttcccaggtcaacctggattcggatgacgtcttcaacctgcgcaagctgaagttcggcgccgaagcccgcgctgctgccggttacggcttctggcagctggcctatggctccaccggcactggcgcataagggggcagctcatggcacgcaaagccgcaactaccgccaagccgaccgccaggctggcccgccaggcggcgggcgatgaagcccagcagagcgtcgagggcatcttcgtgcgcagctatccgccgaccttccggcgtgctggcttcgcgttcaccagcgaaggaatcggaatcgccctgtccgccctgactgaggcgcagctcaaggccatcaaggaagagccccagctgcgcgtcgagtcctgcgagttcttgccggacgacgtcggcggcgagggtgagccgtccgcgtcggacaacgacacccaggaataacccaccccagcagagggaccgcccctggccacggatggccacctattcacaggagttcgacatgagcgaccacaccctggccatcagtcaactgaccatcgccgcgcagaacgccgagcacaacgctccgatcatcgaagcccagggcgacctcgcccaggccgagctggatcgccgggtcgctgccgagtgccatagcgccatcgacgtcctggagcaccaggagcagcaacagtgagctattgcacccaggccgacctggtcgagcagtacggcgaggcgtccatccgccagctgagcgaccgcgtcaataaacccgccacgaccatcgatccggcggtcgtggcccaggctatcgccgatgcggacgcggagatcgatctccacctgcacgcccgctaccagctgccgctggcccaggtacctgtggtgctgaagcgcgtagcgtgcgtgctggcatttgccaacctgcacacccaggtcaaggacgaccaccccgcgatcctggatgccgagcgcaagcggaagctgctgagcggtatttcctccgggaagctcagcctggccctgaccagctccggcaccccggcgcccattgccaacaccgttcaaatcagttcgcaacgcaacgatttcgggggcacctggtgaccactgccgagccgttcgattacctgttcctggagacgctcctggtcgagcgcatccgcgccgaggtgcccggccttcaggacgtttcgggcatccccgatctggccaccctcgacgaacagcgccagggctcgccttgcgtctatgtcgtctacctgggcgacgagatcggcaccggggcgtcgcaccagggcggcagtcgggcgattcagacggtcacccagcactgggcagccgtgctgacgttgtactacgccgacgcccagggcgacggccagggcgcccggcgcgaagccggcccgctgctcggccagctgctcaaggcactgaccggctgggttcctgatcagggcgtcagcccgctggcccgcagcccgcaggcttcgccggtcagctacagcaacgggttcttctatttcccgctggtattcaccgccaactttgtcttcccgaggctcaagtcatggaaaccgtaaaagtcaccatcaccgccgagaaacccaatcacacccatgccggcaagccggtggcgcagggcgacgagatcgaagtcagtcgcgccgatgccgagtttctgctgcgccgtcaattgatcaccaagattcccgccgagcccaaggccgacgagaaacgcgacaagtaacgcgcaacatctgattcctacggaggcctcacatggcacaggaaacgtatttctacgggcaaggtgagattgacgccgcgcctatcgtcaacggcgtcctcggcaaatggcgctggattcaggatgtctcggccatgagcatccagctcgcagtcgagaaggtcgagcacaaggaaagctacagcggccagaaagccctggtccgaagcttccccatcggcaagaccgccaccgtcaacatcaccctacacagcatcggcccggacaacctggcgcttaccctctatggcaaggtcgtggccaaggcggcgggctccgtgacgggcgaggtactccccgccgacctggtggctggcgatgtgatccgattggccaatttcggcgtcagcgaactggtcatcaccgacagcgcgagcagcccggcgcccctcgacccgcagtattacgccctgcgagccgatggcgcctacggcgaggtccaactgctgggtctgccgacgccggccccgacccagccgttcaaagcggcctatgagtacgccgccaccaagcaggtgggcatgttcaccgcgccgcagccgaccgttgccctgcgctataagggcatcaacctggccgaaggcggcgcgccggtcatcgtcgagctgtacaaggtcgcgaccgacccgctccaggagctggcattgatcagcgacggcaacaccgtcgccggtatgcagatcagcggcggaatcctgctggacaccagcaagccggataccggcgacctgggccgcttcggccgcattattcagctggggtgagtcatgaagaagccgaacgacacccccgtcgatgacagcctggaagttctgttccccgaccgtcgtttgacggtcgggggcgaggacctggttgtccgcgagctgaccttcgaagagcagctcacccaccacgcggccctcaaggccattgccgaggcgttcctccaggtgccgcgtgaacacctcgaaggagtcgagggcgcgaccattgcgctggacgtgctgacggagcactggaagctcgttctgccgttgattgccgtgagctgtggcaaggcggatgactgggtccgttcgctccgtccaagcgacggtgagtccctgatgctggtctggtgggcagccaaccagggttttttcgtccgccgcctgtggcgcccgatagtaatggctcaagccctccaacaacgtggggtcgagtcttcgcctgcctcgtcgcagcaggacaccagcgcgaccagctcggccgctacaccgagcgacagctaatgctgttcttccgcgaagccgaggcagaaaagggccgccagcaagcccgcgagctgatggcggtcaaccatggcttcgccgggggctctgtagcgattgccgcctatgatcagctcatgagtaattgaacgtggccgaccaagaccttgtattagccctgcgcatccgcgctgaccttcagcagggtgcggaccaggtcgaggaactgtccgactcgatccaagccgctggcgaccatgccagccaggctggccgggagctgtccggtttgggggagacagccgaccagcaggcggcccggatcaaggcgatggtcgccgcctccctgcaacagcgggaagcgctggacgcgctggcggaaagttcggatcggatgaacaccgccacgcgggcagccaccagcggatggcaggaaagcgcccgtgcgcaatcggcgtcgatgaatgcctaccacaacgccgagcgcgcccgcgaacagcaggttgcagccgagcaacgagcggccgaggctgcggcgaaagccaccgccgagttcgaccggcagcaggccgagctgggcaaactgctcgcggccatcgacccggtcactcgcgagctggagaagctcgacagcctccaggcgcgcctgaatgctgccaggggtcgagggatcgacccggacgtctttacgacctacaacgccaagctccaggagcaacgggaccgcctgctcggcacgtctgacgccatggccgtggccggcatttcggcaggtcagtaccgccaggccatgcggcagttgccggcgcagatcaccgacgtggtcaccagcctggccagcgggatgccactgtggatggttgccatccagcaaggcgggcagatcaaggacagtttcggtggcgtcggcgcgacgtttcaggcgctgggcgatcaggtcaaatcgttcttcgggatcgcgagtaacgccagcgacggcctggacgacatcgcccgaggggcggacgctgccgcagcgtcggccaacaacgccaagacggctatggtaggcctcagcggagcaggtagcgcctttatcgtcatcggcgcggcggtggccgctgcgggcgtggcgctggctctggcgtatgagaaaggcagctcagaggcggacgagctgaacaaggccatcgtcctgaccggcaactatgccggcactaccgccgggcaactgtccgccatggcggcgtcactcgccagggcgaacggcactcagtatgaggccgtggcggtactgtcggaaatcaccgcgaccggcaagttcacggttgaccagatcgagcaggttgccactacctccatcgcgatgcaggaagcgaccggcaaggctgtttcggatacggtcgccgagttctccaagctggccgacgaaccggtcaaagcctcgcaacagctcaacgagaaatatcactacctgaccgcctcggtttacgagcagatagccgccctcgatcagcaaggcgattcgctgggtgccgcccaactggccatggacgcctatagccaggcaatggacgagcgggcgagccagatcgtcgagaacctgggcaccctggaaaccgcttggaagaccgttgccggggtggccaagggcgcctgggacgaaatgctcggcgtgggccggacggagacacccgaggaacgcctggagcaactgaccaaggggcaggccttccagccgggacgcgctgtggccagcggggctgtcttcggcccgttgggctggttcaacgagctacgcaaggcgtatcagcgcagctcgatgtcggacgacgagcgcgggaagcaattcaccgatgccctccaggaaatccaggacgagggcgagaaggcgcagaaggcgcgcctggatcgctacctggaggacgaggcaatacgcggccagcagagcatggacaagctgctggagtcggtgcgcaccaacaaggaaaagcgcgacaagctcaacagggagctggaccggagcattgccgcgatccaggcggctaacccgaacgacgaacgcctgcggccggaaaatatcgccgccgctcgcaaggccatcgatcagaagtacaaagacccgaaaaccccgaaagggccgtctacgcccctcgaccagtccagcgtcaccgaggcgaagaaccgcctggaccagttgcaaaccgatttcaggaacgccgagcagaagctccaggcgcagcagcgcgccggcctgctgagctatgcggactatgtcgcgcagcgcggcgaactgatcagccagaacaaggaccaggtcaccgcagcctatgagggggaaatccaggcgctggaggcgctgcgcgacaaaagttccaccacggcggcccagcgcatcagcctggaccagaagatcgccgaggccaggaacaacatggtcaaggcgcagaagaaggccgacgccgacctggaagtcctccagctcaacgaacaggggcgcctgaagaaacagacccaggcagtcaaggcctacagcgatgcgctccagcaacaacaggatgcgctggccctccagggccaacgtgccgccgctgccgtgggcatgggcgcgcaacagcgccgcctgttcgatcagcgtggcagccttgacgaccgattcgcgcagcagcgcctggacctggcgagccagtatggtgacggctcgcgaggcatgagcctcgacgagtacaacgacaagctcaaggcgctggaagcgaaccatgccgcgatgacccagcagctgcaacgcaactacgccgaccttcaggccgcccagggcaactgggtcaacggggcgacgtcggcgttcgctgactacatcgactccgccaggaacagtgcgggccagacctacgaactgtttagtaacgccttctccggtctggaggatgccgtcgttaatttcgtgatgactggcaaggcgtcgctggatgacttcgtccgcaccatgattggcgacgtggccaggatggcgacccgccagttgggtgcgtctctgctttccgggtttggcctgggcggtggcaccgatggcggcgcccaggggctgacggtcggcgcctcggctgtatccgcctcggccggcgccctggcgactgctggcggcacgctcctgagcggcgccgccgctatccaggccgctgccgcctcgctggccgccgccaatggtgtcagtggggtgaccggcgctgcgggagctgccggggccgctggagtggctggtggcgggagtggctggttgtcttcgatcaccagcattttcggcttcgcgggtggcggccaggtccagggaccaggcacaccgaccagcgacagcataccggcctggctatccaacaatgaggtcgtaatccggtcggcatccgccatgcagccagggcttactccgctgctgctggacgtcaaccaacgcggatgggcggcgctacatgactgggcgggggccgtccgccacgccaccgggggcgtcgccggcattcccgcgccatccctaccacgcccaagcatgggcgccgctcagatacaggaaccgtccaagaacttcagcgcatcagtctccaacgcggtccacctccatgctgttcaagaccccgaccagatggcggccgacatgtgggccggcaagggcggcgaccattacatcgtctggctgaacaagaaccgccaggccgtcaagcaaatactcggaaactaggaattcatggctactgaaatcggcaccgccacgaaccaccagaacctggtcgagcgcctcgtccagttcctcaccgcgaacccagacctggtcgcggctggccaggcctacgagaaggttttcgacaacaccatccccgcgaccggaacggccatcgccgtgcgccaggtgacactgcgcgccccaggcctgggcggcaccgatagcatctacatggggattcagagttacggcgatacggcgctggactactacaacctgcgcctgatgggcggcacggcgtttaatcctggagcgatcccgcccggtggcgactactggaccgcgtttgccaactacagtccgcgggtccaggcgctgctgtggaaccagcccatgccgtactggttcttcgccaacggccgacgcttctgggtcgtcgtgaaagtctcgacgatctacgagtcggccggcgccggcttcatcctgccaccctgtccaccgtcgcagtacccgtacccactcgctgtagtcgggtcgtatcgcggggacgtcgctgtgcgctggtccgatgtgagtgaccggcaccgaggcatcagcagcccctacgagcgaagctgctatctccgcgatcccgccgggcgctggctcggtttcactgtagacggaggggctgccaacgagtccgactacaacaatcggacgctcctcccgctgggctgcggccgttatgcgggcagcagtgacaccgtggtcaaacaactgcgggattcattcgggaagttcccgctcaaggcgctgtcgttcgtcacccgcgaaaccgagggtcgccgaaacctgggcgacttcgacggcgctttctacgtgccaacgctcaactccggcgccgaggacgtgattgtcgaggacggagtggaccacgtcgttttccaaaccgcttggcgtagcggtaacccttggctctacgcgatcaggaaggactgacatggcctatttcacaggaacagctaacaacccgtccgacctgctcgccaaactgcgcgtccacgccgagtcgctcggctgggtcaccgaccgcgcctcggcatcggaatggctttgtcacaacgctgatgggtactggtcattcaatgccggagccaatcagttccagatggcgggcaatacggggttcgataacagcctggcgtggaacgcgcagcccggtaactcggtgcagaacaacccttattcgtcgaagggaccgaccgtcgcacagctgagcggtgggccgttcacgcgctaccacctgtttgccacggcggcctatctgcacctgcacgtcgagattgcggccggtcagttccggccggtgatgattggctcgctcaacaagcgcggagtcgaatacagcggcggccagtacgtatgcggctccgtaatctatcagtcgggccagatgctgacatcgaactggtcctgtcatccgttcgacggctatcacgttcgctatagcgacggtggttgcgtactgcgtgtggatggcctggatggcggcccctcgcccgactggctgccattcgactacgcgacgaacatcccccggcgggtcatcgggccaggccgtggaaactacagaagtcagtaccatcctgacgtcggactgatcgacgccagcgcaaacgagctgaacagctcgaccaccactgtgccgtgcgccatctatgcgttcggcgctcagcagcgctcgcgctacgtgggcgaagtgccggactttggcatatgcaacatggccttcctcgcgcctggtgatccgcttgtcgtcggtagcgacacttggcgcgtatatccgttgctccaacgcggaaccgctaccgacttcgacagcgccagcgcctgggtcggctattgcttccgggtggtcgagtgatggcgacgtttccagggttccaggtgccgaagcctgtggaggggatcgttgccggcatcacgccgaatatcgacgccctggagctgaaccaggacatcagccttgcagcggtcgcggcctcgacctgggccggcgcctatggggcgcatcagccggtagaggtgatccattcgacctaccaggctgtccaccaaagcgctctggaagagaactactacaaccgcctctggttgattccgaccgcaatggaactgggcaacgtcgtcagcacccagatacgaccggcatcagtctggaacgcttatttcagcccgcgcatgctgaccgctatcgaccgcgaagccgcagacggcattacgctgtccggccaggcttcgccgccgctgggttttgccgccctggaggaacgcacctggacggtcagcattggcaccgacggcccgcccgtagtcaatgcccgaatcatctggaggctccagggcgagccggacctggccctggtcatcactggcaatcgcatcatcgcctggacgttcgcgccggactggggcgacagcatcgtcgaacgcctgagcgcatcgacaaatatcctgcaaagcgaatcggccgtgacccagcgccgagccatgcgcctggcgccgcgccgagagttcgaagcgaacatgtacgcggtggatcgcgagcggcagttgctggacatgacgctgttcggctggggtgcgcgcatttgggcgctgccgatctggcctgatatccagctgctccaccagccgctggcggccgggtcgctgagcattccgtgcgacacggccggcctcgacttccgcgacggcggtctggcgatgctgcgcggcgaggacgcttttacttacgaggtcgtcgaggtcaagacggtgaccgccagcggcctggacctggtccggcccgtccaggccgcgtggggaactggctcgcgactgtacccagtgcgcaccgcgcagctgaccgaacagcccacgctgacccggctgaccgataccgcgcagtctgcgcgggtgtcgttccttgtgatggaacccagcagttggcccgaggtgatgccggcgacgatgtaccgggggcgtcctgtcctggaacagcgccccgacgaaagcgaagacctgacgtcgagctatcagcgcctgctgtccaccctggataacggcagcgccattccccgcgtgaccgacgtcgccggcatggcgctgcccgtcatcggtcatcgctggatcggcatgggccgagccgaacggtcggcgttccgcagcctggtctatgcgctacgcggccagcagaagccgctgtgggtgccgacccacgccgacgacctgaccctggtcgccaccgtctcgcagctgtccaccgcgctggacgtgcgcaatatcggctatgcccgtttcgccaacggccggccgggccgtcgcgatatccgcatcgagctatacgacggcacggtctatcaccgccgcatcctcaccagcacagagctggacgccgacaccgagcgcttggccatcgatgccgccctgggccggctggtcgagcctggtgacgtggcgcgcatttgtttcatggcgctctgtagcgccgccaccgacgtggtcgagatcgagcacgtcactgatagcgagggcgtagcaactgccgccctgacgttcaaaggggttcgtgacgatgagttttaacagccgcgaaagctcgctcgcggatgggcagccggtgcggctgtaccagttcagccgtggagccatccgctggagctacaacagcagcgaccgggacatcacctaccagaaccagattttccgcaccgtgccgggcggcatcactgacaacgggatcatctgttccggcgatccgcagtccgaccagttcgtcatcaccgcgccggccgacctcgacgtcgcgctgctgtacaagacccggtcgccgagcggtgccatcgacttggtcgtctacgacatgcactacggcgacaccgaggcagcggtttcctgggtgggccagattggcgatgtggactggccgaccgtggacagctgccgcataacgtgcgtgtcagaagacgaactgatggaccagcccggcttgatcgacacctactgccgcacctgcacggcagtcgttggcgaccatcgctgcaaggtcaacctcgttccgtatcgcgtgacgctgacgccgcagagcatcagcggctgggtgatctccagcggcgtggtcgccggctatgtcgatggctggtttaccgggggctacgtcgagtggcaagtggacggcgacaactacgatagccgctacatcgagcggcacgccggacccgatctttacatcctgggcggcaccgagggcattccggcaggtggccaactgcgggtttatccaggttgcgacgggctcgcgcagacctgcgacgacaaattcagcaacctccccaacttcagggggtttaacgcgatgcaaggcaagtcgccattcgatggcgaccaggtctggtgaggtaggccatggacccgatcacaatcaatctcgtcatcctggcggcgtcgttcatcctatccaaggtcttggcgccgaagccgcagaagcccaagccgaccgcctttgaagacatcgatttcccgcgctgcgacgagggtgacgaacaggtcgccgtcttcggtcagtgctggtcgaagagctggatggtgctgaccgtgggcaactacagaacgaaggcgatcaagaccaaagggagcaagaaatgatcgttacggctcagcacctgcataccgtgccgacctggaccactcggcagggctactgccaccggcaggcgcgggacttcttcaagcgccatggcctggattggatggcgttcttacgggacggcatcgaggccgacgtgctagtcgcgaccggcgacgcgctcgcgctcaagctggttgagcacgcatgccaggaggtagccgatgggcgctaaaccgaaggcacagacggtcgggttcgagtacttttttgacatccatttcgccctgggtaagaagatcgacgaggtctgtgcaatccgggcgagcggcaagaccgcatggaagggctcgatcaccagtaacggccaggttcgcatcaatgcgccggacctcttcggcgggaagaagggcgaaggcgggctcgacggaacgcttgacgtgctgtttggcgaggaggaccagggcgtcctgccgcgcctggcggcgatgctcggcggcctggtaccggcgttccggggcgtcaccacgtgcttctattccggcctggtcaccgccatgaacccctacccgaagacctgggagattctgcgccgaggcggcaaccgcctgtgggacggcaacccctggtatcccgaaaagcaatttatctggctcgcggacggtcagatcaaggcgatgaatccggcgcatatcctctatctcgtctacaccggccgggacttccgggggctggcccgcacgcggatggacgaggcgagctggcgggccgctgccgacaagctgtatgccgagggtttcgggctgtgctttgaatggaccaggtccgacacgttctcaaacttctgcgagacggtgaaatcgcatatcggcgccgaggtttacccgaaccgacagaccggacaaatcagcatccgcctcctgcgggacgactacagcgttgcagacttgccgctgttcgacgaggacagcggcctcctggagatcacccaggagaagaccggctcgacctcgctcgcgccgagccagcttatcgtcaagtacatcgaccagaccgacggcgcgcagcgccaggtcatcgtcaacaacaacgcggtcgccgcttcgcaggggcggcggtcgtccgaggaagtcgagttcctgggcgtgccgactggcgagctggccgggcgagtcggggagcgggaaatgcgtctgaagacaaccggtctgaagcgctataaaggcgtattcgaccgccgcgcccgtagcctgaaccctggccagccgttccgcatccgttcgacccggcgcggcatccctgaaaccgtcgtccgggtcggccggatcgaggacaacttcctcggcgacggcaagatcaccctgaccgtcgtccaggaccagttcaatctgccggcgactaccggcgtggcaccaccgccaccaggctggaccccgcccgaccggacgcctcgggcggtcaccgtgcggcgtctgatcgaggcgccatatcgcgaactggccggcgtgatcgatccggcgaatctccagctcctggacgtgtccgcctcgtatcttgccgccttggccgaggcgccgacgagcctgtcgcagagctacaccttgaccgaccgcgtcggcagttctggcgcgttcgttgatcgcggaaccggcgactggtgcccgaccggactactcgccgccgagctgccgctggcggccggcccgaacgtcgtcacgttgacgaacgccacccggctggaggacgtcactgtcggccaggccgctgtggtggacgacgagatagtccgggtcgatgccgtcaactatgccagtggcaccgtcaccctggcgcgcggctgcgccgataccgtgccggccaagcacttggccggggctcgggtctggttctacgacacgttcgaagcggtggacgagacggtatacagccagggcgtgacgctccaggcccggctgctgaccaacaccagcgagggccagctcgccccggcgctggccgccaccgacagcctcactctgaccgggcgccagggcaagccgtatccgcccggccagttccgaatcaacggcagcgcgtacccgacgaaggtctacggagcgctgtcggtgagctgggcgatgcgcgaccgcatcggccaggccgaccagttgatcgataccactgtcggcaacatcgggcccgaagatggggcgacggtgacgctccaggtctacagcggcacgacgctgaagcgcacctatgccggcctcacatccagtaactggtcctatccgctggccgaagacgtcgctgacggcctgctccaggacgtgcgcctggtcctgcgcagcgtccgcgacggcatccaatcctggcagcaacacgacatcaccatcgaacgacacggcctgggcttccgcttgggcgaagaccttggaggcgtttccgcatgactctctatatggggcctaacaccggcctgctgatcaacggcttgccgggagaagggcattacagcgatctgattcggatgtggcgctgggatgactttctgaggcagccggtcgtcaaggggcgcgtcgccgcgctcccgaccagcggccaggccgagggggacacgtacattttcactggctccggctccaatcagaaccgcctggcgcgctggtgggcaacgggcgccaccacggcaatttgggagtacatgccgccacggctgggctggcgtgtccaggtcgcaaacgagacgacgccgagcggccaggtcaagacctacgagtattccggcacggcgtgggtcgagctggtgggcggtatgtcggacgcgcccagcgacggcagcaactacgcacgcaacaacgggacgtgggggaagctgggaaccgctgccggagcagacctcaacggcatgccgttcctcaatctgatgcccgatagcgggcggtacgcaggcagtatcaacccgctaatcctgcgattcactgaggctttttccagtacgttcctgacgccatggaatggcgcgtcaatcgctgacggcggaaagtacatctacgacaacaccacaaacggcgggacggctggcaatctgaatcaacgtgtccaggacttactggtggcgatggggcggtccagtggcagcttggcgcgctatggcgtggagttctataccgctgtgctgaccgctggccccaacgcaacgaccggctctacgggcgccgacggcacgacccgttatctccagatgacgaactcgtcgagggcgctcttcatcgccaacggctggtgtactgcggttctttggatacgcgcggaggccggatcgcttcacttcatgccggcaacggccccgacgactgactacaagatatggctgaatggtgcgcctgtactgccggggcaggtgctgaccccggccgatggatggaagcacgtcaggatttccaaaaagagcgcgcagggctacgacaacggcttcccgttcctctatatgtcgctgggcgccagtgcagctatggcctgtccggcattcttcggcggcctggtcgatcccggcatccacgtcgcgcctattgcaaccgtcaactcacagagcgcatgacaatgacgaaacgagttctattgaaaggcgagttcttcgcagagtgggccggctcgctggacgaggccgccgcactcgctggcgtcccggtcggcgacctggcgttccatcccgacgacctcctggccgaagtccaggagttacgccgccaggcctatcgcaccgagtccgacccgctgcgcctggaggccgagtttgacgccatagccgctggcaccgagccggacctggaggcatgggtcgcggccgtgcaggcgatcaaagaacgctatccgctcccccagtcctaagcgttttgataattgtgaccaacgtcgcctttttgctacggtccctagctgatgtgcggagtagatagggaagttggtatggacgaggtgcttagacggaggctgcgggcggagctgctggaggtggggtttctcaaccagtgctgccttgatctgatggatgcgatggaggccgagttcagcctcaccgaggaccagcgcgaatgcatcgagcagctcggccgattcctgcgggagggcatcggcaagctgaccgctctgtctgagcgggtagccgatggcgatatcgtcgtgctgtgctgatcttttgaaattcttttgccgctggcgaaacggttagggcgcgtcatttattgcgcaaatccgcgccaaatttcgcgcccggtacaggcc